(19) United States
(12) Patent Application Publication
(10) Pub. No.: US 2023/0172976 A1
(43) Pub. Date: Jun. 8, 2023

(54) ANTIMICROBIAL MATERIAL

(71) Applicant: COPPER CLOTHING LIMITED, CATERHAM SURREY (GB)

(72) Inventor: RORY DONNELLY, CATERHAM SURREY (GB)

(21) Appl. No.: 17/998,936

(22) PCT Filed: May 21, 2021
(86) PCT No.: PCT/GB2021/051245

§ 371 Date: Nov. 16, 2022

(30) Foreign Application Priority Data
May 22, 2020 (GB) 2007672.5

(51) Int. Cl. A61K 33/34; A61K 33/30; A61P 31/02; A61Q 19/08; A61K 8/27; A61K 8/19

(52) U.S. Cl. CPC A61K 33/34; A61K 8/19; A61K 8/27; A61K 33/30; A61P 31/02; A61Q 19/08; A61K 2800/412

(57) ABSTRACT

The present invention relates to an antimicrobial composition comprising copper and zinc incorporated into or coated on a substrate material, wherein the substrate comprises a dermatological composition for external use on a subject. The invention also relates to methods of making the described antimicrobial materials.

### FIELD OF INVENTION

The present invention relates to an antimicrobial composition comprising copper and zinc incorporated into or coated on a substrate material, wherein the substrate comprises a dermatological composition for external use on a subject. The invention also relates to methods of making the described antimicrobial materials.

### BACKGROUND

The antimicrobial properties of certain metals have been known for a substantial period of time. This unique property has been capitalised on in various industries, including agriculture and healthcare, in an attempt to control infection and contamination.

One metal commonly used in the healthcare setting is silver. The antimicrobial action of silver is dependent on the biologically active silver ion, resulting in irreversible damage to key enzyme systems within the cell membranes of pathogens, resulting in cell death. The most effective conditions for silver to act as an antimicrobial agent are those with higher temperatures and excess moisture. These conditions aid the ion-exchange reaction required for the release of silver ions. However, these particular conditions are rarely replicated in day-today healthcare settings, therefore limiting the efficacy of silver in controlling infection rates. In contrast, copper has been shown to display impressive antimicrobial efficacy in a broad range of environmental conditions.

Copper based materials are used in a wide-range of products, including wound dressings, sanitary protection products, toilet seats, clothing and footwear. Additionally, copper based materials are used in a number of medical settings, including in the treatment of arthritis and osteoporosis.

Copper is known to exert its actions in a number of ways; acting as a biocidal substance, enhancing microcirculation and reducing tissue inflammation at the site of injury. Additionally, the antimicrobial properties of copper are known to be an inherent feature, therefore representing a cost-effective and long-term solution to reducing infection rates.

The interest in using antimicrobial materials as an infection control method is particularly prominent. A particular challenge is the prevention of spreading infection, either between individuals, or between individuals and commonly used surfaces. The spread of infection in this manner can be extraordinarily fast and poses a significant risk to vulnerable members of the public, for example, the elderly or those with compromised immune systems or underlying health conditions.

One current method employed as an infection control method includes the use of alcohol gel sanitiser. However, such products are known to be ineffective against a range of microorganisms, the effectiveness of the product can be dependent upon the volume of product used, and such products may evaporate quickly off the skin of the individual. Additionally, it is known that the alcohol content of these products may strip the skin of the outer layer of oil, resulting in negative effects on the barrier function of the skin.

The consequences of not preventing the spread of an infection are manifold. These include enhanced hospitalisation rates, long-term disability, a reduction in workforce and an increased economic burden on society. Copper based materials have been shown to enhance the rate of wound healing via the mechanisms previously outlined, and as a result, increase the resolution of various infections. Additionally, silver based products have been reported to display much higher levels of toxicity compared to copper based products. For example, silver has been shown to lead to renal toxicity following topical application. However, the form of these copper based materials has varied widely, including the use of various copper alloys and copper salts.

Copper salts have been used for their antimicrobial properties in wound dressings. For example, U.S. Pat. publication 2016/0220728 describes antimicrobial compositions comprising surface functionalised particles of low water solubility inorganic copper salts, or such copper salts infused into porous particles, and their application of the compositions for wound care.

Antimicrobial properties have also been associated with a copper-tin alloy. European patent publication EP 2 476 766 and U.S. Pat. publication 2013/0323289 both describe antimicrobial raw materials comprising a substrate layer and a copper-tin alloy layer disposed on the substrate layer, suitable for use as wound dressing films and adhesive bandages. However, a number of issues are associated with this alloy, including skin discolouration when used in the context of a wound dressing.

Copper salts differ substantially to alloys in terms of the type of chemical bond involved between the two components. Alloys are produced via metallic bonding whereas copper salts are a result of ionic bonding between a base and an acid.

Copper based materials often involve an additional component, as opposed to using pure copper in isolation. Pure copper is a soft and malleable metal, limiting its utility in healthcare, agricultural and engineering industries. Conversely, copper alloys confer a number of desirable properties, including increased resistance to corrosion and enhanced strength. The increased resistance to corrosion and enhanced strength results in a more cost-effective and long-lasting material with wide-reaching applications in agriculture and engineering but such properties are not associated with advantages in healthcare applications. Copper takes on different properties when combined with different metals. For example, a copper-tin alloy results in a more brittle product compared to a copper-zinc alloy.

There is a need in the art for improved antimicrobial compositions that can be used to reduce the incidence and spreading of infection caused by harmful microorganisms. There is also a need in the art for infection control products that minimise negative side effects for the end user and products that provide additional beneficial properties alongside the antimicrobial properties of said product.

The listing or discussion of an apparently prior-published document in this specification should not necessarily be taken as an acknowledgement that the document is part of the state of the art or is common general knowledge.

### SUMMARY OF INVENTION

The present invention provides a novel way in which superior levels of infection control can be implemented in both a healthcare and domestic setting by combining the advantageous properties of common skin care preparations with the antimicrobial properties of chemically bonded copper and zinc. The combination of these features are perfectly suited for application in a range of products intended for personal use to minimise the spread of potentially harmful microorganisms. The inventors have surprisingly found that the antimicrobial composition herein disclosed provides a longer lasting, broader and superior protection to those products already available. Additionally, the inventors have found that the herein disclosed antimicrobial composition has the additional advantage of being an effective anti-aging agent/angiogenesis promoting agent. Accordingly, the present invention has multiple benefits derived from a single product or composition, which can be used by an individual on a regular basis.

In a first aspect, the present invention provides an antimicrobial composition comprising a substrate and a metal component, wherein the metal component comprises chemically bonded copper and zinc and wherein the substrate comprises a dermatological composition for external use on a subject.

Accordingly, the present invention provides for an antimicrobial composition suitable as an infection control agent, anti-aging agent and as an agent for promoting angiogenesis.

In a second aspect, the present invention provides a method of manufacturing an antimicrobial composition comprising a substrate and a metal component, wherein the metal component comprises chemically bonded copper and zinc, and the substrate comprises a dermatological composition for external use on a subject, the method comprising the following steps:
a) combining copper and zinc to produce said metal component;
b) heating the metal component to a molten state;
c) disrupting said molten state with a high velocity gas, and;
d) combining the metal component with a substrate.

### DETAILED DESCRIPTION

The following description is presented to enable any person skilled in the art to make and use the invention. Various modifications to the disclosed embodiments will be readily apparent to those skilled in the art.

In a first aspect, the present invention provides an antimicrobial composition comprising a substrate and a metal component, wherein the metal component comprises chemically bonded copper and zinc and wherein the substrate comprises a dermatological composition for external use on a subject.

The term ‘antimicrobial composition’ refers to a composition having antimicrobial properties, for example biocidal or biostatic properties. In the context of the present invention, the term ‘biocidal’ is understood to mean a substance that can destroy, deter, render harmless or exert a controlling effect on a pathogenic organism, whereas the term ‘biostatic’ refers to a substance which can inhibit the growth or multiplication of an organism, for example a microorganism. It is envisaged that the present invention will be useful against any microorganism, for example any bacteria, virus and/orfungi. In particular, it is envisaged that bacteria in the Genus Staphylococcus and Klebsiella, fungi in the Genus Candida, and members of the Coronaviridae family, for example, COVID-19, will be sensitive to the presently described materials. Accordingly, the present invention may be described as being anti-septic, anti-bacterial, anti-viral, anti-fungal, anti-pathogenic or anti-microbial.

The present invention provides compositions with surprisingly high antimicrobial activity. Accordingly, in one embodiment of the present invention, an infection control agent is provided. Due to the high level of antimicrobial activity seen with the claimed composition, the efficacy of the product is less affected by differing volumes of the product used compared to already available products, for example, alcohol gel sanitiser. Dermatological products, incorporating the compositions of the invention will reduce incidences of infection by helping control the spread of the infection causing microoganisms. The present invention is particularly useful in the prevention of infections associated with human to human contact, such as handshakes, or surface to human transmission, wherein microorganisms present on objects, for example, door handles and taps, are transferred to humans due to contact with said objects. It is envisaged that the present invention may be particularly useful for application on commonly exposed areas, for example, an individual’s extremities, in particular their hands and/or forearms. Accordingly, as well as reducing spread of an infection, it is envisaged that the use of such a product may be efficacious in treating an already present infection, for example, athlete’s foot infection or treatment of a nail fungus.

By ‘substrate’, we intend any suitable structural material to which the metal component can be incorporated, thereby providing a physical medium on or in which the metal component may be deployed. The substrate of the present invention comprises a dermatological composition for external use on a subject.

Preferably, the dermatological composition for external use on a subject may be a cream, gel, lotion, spray or ointment. These dermatological compositions may also be described as skin care preparations or topical compositions/preparations. The skilled person will understand that the form the product takes may depend on the desired consistency, desired use and skin-type of the end user. These skin care preparations are advantageous over alcohol based products, due to the slower rate of evaporation, providing for a product which is active for a prolonged period of time.

By ‘chemically bonded’, we intend any lasting attraction between atoms, ions or molecules of copper and zinc as a result of ionic, covalent or metallic bonding. Accordingly, this may include copper alloys or copper compounds, including but not limited to copper salts and oxides.

Preferably, the metal component of the antimicrobial composition comprises a copper-zinc alloy. An alloy is understood to be a mixture of two elements, one of which is a metal. In this instance, the copper-zinc alloy is understood to be a substitutional alloy, whereby the atoms of the two components may replace each other within the same crystal structure, creating a sea of delocalised electrons.

A skilled person would recognise that in order to produce the required alloy, elemental copper and zinc are mixed together in their molten form before solidifying as a new and distinct chemical entity. In one embodiment, it is envisaged that additional metals and compounds thereof, e.g. salts, may be incorporated into the material or metal component. These metals include, but are not limited to, tin, iron, lead, zirconium, copper, zinc, silver, gold, palladium, platinum, iridium, aluminium, nickel, tungsten, molybdenum, tantalum, titanium, iodine. It is understood that the latter compounds may be additional components to the claimed material which contribute to a further enhancement of the antimicrobial properties of the material.

The use of an alloy, as opposed to the pure form of the metal or associated compounds, results in a number of advantageous properties compared to the use of pure copper. Specifically, a copper-zinc alloy benefits from the extra antimicrobial properties of zinc, excellent malleability/castability and high strength.

The particles of the metal component are expected to measure between 10-80 µm, with the preferred size being anywhere from 15-30 µm. A finely ground powder releases more ions compared to a course powder, the released ions of which may be responsible for the antimicrobial effect.

It is envisaged that the metal component will contain at least 60 % copper. This formulation will have enhanced antimicrobial properties. Preferably, the metal component comprises 75-80 % copper with a corresponding amount of 20-25 % zinc. As outlined above, the metal component may additionally contain other element(s), compounds and salts thereof. These additions may confer beneficial properties to the claimed material. For example, additional components may further enhance the antimicrobial actions or allow for increased longevity of the claimed product.

In one embodiment of the present invention, the metal component may be interspersed throughout the substrate. By ‘interspersed’ we intend that the metal component is scattered between particles/molecules of the substrate material. Such a configuration could alternatively be described as ‘impregnation’. The metal component may be evenly or unevenly dispersed throughout the substrate material. A skilled person would understand that the degree of interspersion, dispersion and/or impregnation may depend on the base skin care preparation to be used and/or the process used to apply the metal component.

It is envisaged that the substrate may comprise an emollient, an emulsifying agent, a petroleum-based agent, a thickening agent or a moisturising agent. The substrate may yet further comprise a humectant, an antioxidant, an essential amino acid, a preservative and/or a fragrance agent. Examples of humectants include but are not limited to salicyclic acid, glycerin, hyaluronic acid, urea, panthenol, sodium lactate and glycol. Examples of antioxidants include but are not limited to vitamin C, vitamin A (retinol), vitamin E, resveratrol, CoEnzyme Q10, niacinamide, polyphenols, flavonoids and glutathione. Examples of essential amino acids include but are not limited to arginine, histidine, methionine, lysine, proline, leucine and glycine. Examples of preservatives include but are not limited to parabens, phenoxyethanol and organic acids. The exact composition of the substrate will depend on the desired end form of the product, for example, if it is a lotion or a gel. The inclusion of other elements, such as antioxidant and fragrance agents, provides for a product, which is not only antimicrobial but has numerous other beneficial properties in a cosmetic setting. Accordingly, the present invention also provides for a cosmetic product comprising the antimicrobial composition disclosed herein. By “cosmetic product”, we intend any product used to restore or improve an individual’s appearance. The inventors are unaware of a product that is able to combine impressively high antimicrobial properties with such products.

Preferably, the substrate may include the following ingredients following manufacture :
a) Sodium carboxymenthylcellulose
b) Surfactant
c) Glycerine
d) Citric Acid
e) Any of the aforementioned components described above or mixtures thereof.

Preferably, 2-6 % of the substrate by weight consists of the metal component. For example, 2-2.5 %, 2-3 %, 2-3.5 %, 2-4 %, 2-4.5 %, 2-5 %, 2-5.5 %, 2.5-3 %, 2.5-3.5 %, 2.5-4 %, 2.5-4.5 %, 2.5-5 %, 2.5-5.5 %, 2.5-6 %, 3-3.5 %, 3-4 %, 3-4.5 %, 3-5 %, 3-5.5 %, 3-6 %, 3.5-4 %, 3.5-4.5 %, 3.5-5 %, 3.5-5.5 %, 3.5-6 %, 4-4.5 %, 4-5 %, 4-5.5 %, 4-6 %, 4.5-5 %, 4.5-5.5 %, 4.5-6 %, 5-5.5 %, 5-6 %, 5.5-6 %. The inventors have surprisingly found that this range of the metal component maintains all the desirable antimicrobial properties of the product, produces a product with long-lasting and reliable effects and is economically viable.

Also envisaged is the inclusion of further additives to the material to improve the antimicrobial properties, if required. These additives may include chelating agents, magnesium sulphate and/or a copper peptide. These additives may be incorporated into the substrate at 0.1 to 1 % by weight, for example about 0.5 % by weight. The term “chelating agent” is used to describe a substance that can form several bonds with a single metal ion thus forming a more stable complex. A skilled person would recognise the action of such substances could enhance the antimicrobial properties.

The present invention may be effective when it comes into contact with any contaminated surface. In a preferred embodiment, the present invention may be used as a sanitising product for external use on a subject. In yet a further preferred embodiment, the present invention may be used as a hand-sanitising product.

The present invention provides a high level of antimicrobial activity and therefore has wide-reaching applications. The present invention includes an infection control product comprising the antimicrobial composition of the invention. Such a product may have utility in the healthcare setting, as a sanitising product or in a domestic setting, for example, as part of maintaining hygiene as part of a daily routine or after coming into contact with particular surfaces. By ‘infection control product’ we intend any product that treats, prevents or attenuates the development and/or spread of infections.

The present invention also provides for an antimicrobial composition for use as an anti-aging agent or as an agent for promoting angiogenesis. As a result, the present invention may be used to treat the symptoms of aging and/or improve the appearance of the skin or aging associated skin conditions. The present invention therefore provides for a cosmetic product used for restoring or improving an individual’s appearance. For example, the antimicrobial composition may be used to prevent, reduce or delay the formation of wrinkles, loss of skin tone and elasticity, for the formation of pimples and blackheads. The antimicrobial composition may also be used to prevent, reduce or delay the formation of skin discolorations, such as brown spots, age spots or liver spots, rejuvenate dry or irritated skin, close or tighten pores, improve skin texture, smoothness or firmness and create smooth and supple skin with improved elasticity. The latter conditions may be further improved by the present invention also being an agent appropriate for promoting angiogenesis. The present invention therefore provides for an antimicrobial composition for the treatment of aging by promoting angiogenesis. The present invention also provides for an antimicrobial composition for the treatment, prevention, reduction or delay in symptoms associated with aging by promoting angiogenesis. Such symptoms may include wrinkles, loss of skin tone, loss of elasticity, formation of pimples and blackheads, skin discolouration such as brown spots, age spots or liver spots, dry skin, irritated skin, open pores, rough skin tone or reduced elasticity of the skin. The term ‘angiogenesis’ is understood to mean the physiological process by which new blood vessels form from pre-existing vessels. This process has been suggested as a method by which the symptoms of aging may be treated/improved/reduced/delayed. Thus, the present invention provides for an antimicrobial composition having numerous advantageous properties in addition to its antimicrobial properties.

In a second aspect, the present invention provides a method of manufacturing an antimicrobial composition comprising a substrate and a metal component, wherein the metal component comprises chemically bonded copper and zinc and the substrate comprises a dermatological composition for external use on a subject.

The method comprises the steps of a) combining copper and zinc to produce said metal component; b) heating the metal component to a molten state; c) disrupting said molten state with a high velocity gas, and; d) combining the disrupted metal component with a substrate, wherein the substrate comprises a dermatological composition as set out herein. Preferably, the dermatological composition is a cream, gel, lotion, spray or ointment.

Thus, one method of producing the metal component of the invention may involve a plasma or gas atomisation process. It is envisaged that powdered forms of the metals may be used in the method of the invention but other forms could be appropriate as would be understood by a person of skill in the art.

It is envisaged that the plasma or gas atomisation process will result in a powdered form of the metal component, which can be combined with the substrate as appropriate, as would be understood by a person of skill in the art.

In a preferred embodiment, prior to the commencement of the plasma or gas atomisation process, the metal component may optionally be reduced in size via the use of a mechanical attrition process. By ‘mechanical attrition’ we intend any process by which the result is the gradual breakdown of the metal component into smaller elements. This process can be achieved via the use of a number of attrition devices, including but not limited to: attrition mill, horizontal mill, 1D vibratory mill, 3D vibratory mill and planetary mill. All of the above devices result in a reduction in size due to the energy imparted to the sample during impacts between the milling media. Thus, metallic forms copper and zinc may be ground down to an appropriate form for us in the methods of the invention.

Once the copper and zinc have been combined, the atomisation process may proceed. As would be understood by a person of skill in the art, the means of combining the copper and zinc may differ depending on the atomisation process to be utilised.

Plasma atomisation requires the metal component to be in a wire form to be used as a feedstock. This is typically a wire of an alloy of the metal component, as would be understood by a person of skill in the art. Contrary to conventional gas atomisation, plasma atomisation uses plasma torches to instantaneously melt and atomise the wire in a single step. A cooling tower is then used to convert the droplets formed into a spherical powder.

Alternatively, conventional gas atomisation may be used. This may involve the heating of the copper-zinc metal component to approximately 2000° C. to produce a molten state of said component. By ‘molten state’ we intend the liquid form of said metal component when exposed to high temperatures. As would be understood by a person skilled in the art, a high velocity gas stream may flow through an expansion nozzle, siphoning the molten metal component from an input chamber. Examples of gases that can be used in this process include nitrogen, argon, helium or air. The skilled person will recognise that it is possible to use more than one gas in this process and the preferred gas or gas mixture will be inert/unreactive. The choice of gas used will depend on the desired end disrupted metal (powder) characteristics. To provide a suitable metal component for use in materials of the invention, high velocities of inert gas may be required. A skilled person will recognise that the velocity required will differ depending on the gas used but are likely to be within the range of 100-2000 m/s. This process disrupts the liquid stream of molten metal and results in the production of fine particles, culminating in the desired powdered form of the metal component. Obtaining the powdered form via the above methods has a number of advantages; production of highly spherical particles, low oxygen content and adaptability to the production of copper and zinc. A skilled person will also recognise that alternative methods of producing the metal powder may exist which could be employed to achieve the same effect.

To produce the final antimicrobial composition, the metal component is added to the substrate. Specifically, the metal powder is added in small quantities until the entirety of the product is transferred to the substrate. The resulting composition is mixed at room temperature (20-22° C.) for 2 hours at 350 rpm,

The present invention also provides a method of preventing or treating an infection comprising utilising the antimicrobial material of the invention in a medical or veterinary setting.

In order that the invention may be more clearly understood embodiments thereof will now be described by way of example.

### Example 1

Test of the antimicrobial composition on two different strains of bacteria: Staphylococcus aureus and Klebsiella pneumoniae.

Each test organism was prepared to approximately 1x10⁵ colony forming units (CFU)/mL in 0.85 % NaCl. For each sample, five replicates were inoculated with each test organism. The inocula were enumerated using pour plates of Tryptone Soya Agar (TSA) at the point of inoculation. The inoculated samples were held for 24 hours at 24° C. ± 1° C. and >95 % humidity. Following the exposure time, the inoculated test pieces were aseptically removed to 9 ml diluent. This was vigorously shaken to ensure thorough resuspension of any remaining test organisms. The resulting suspension was plated out in TSALT (TSA supplemented with 0.3 % soya lecithin and 3 % Tween 80). Plates were incubated at 31° C. ± 1° C. for at least 5 days.

TABLE 1

Effect of 0 % CuZn material on two types of bacteria: Staphylococcus aureus and Klebsiella pneumoniae.
24-hour contact time

| Sample: | Replicate | Recovery per test piece (CFU) | | Log₁₀ reductions | | Mean Log₁₀ reductions | |
|---|---|---|---|---|---|---|---|
| | | S. aureus ATCC 6538 | K. pneumoniae NCO9633 | S. aureus ATCC 6538 | K. pneumoniae NCO9633 | S. aureus ATCC 6538 | K. pneumoniae NCO9633 |
| Inoculum (zero time) | - | 1.1 x10⁵ | 9.6 x10⁴ | - | - | - | - |
| 0 % CuZn | 1 | 5.0 x10³ | >1.0 x10⁶ | 1.34 | 0 | 1.32 | 0 |
| | 2 | 5.6 x10³ | >1.0 x10⁶ | 1.29 | 0 | | |
| | 3 | 4.7 x10³ | >1.0 x10⁶ | 1.37 | 0 | | |
| | 4 | 5.7 x10³ | >1.0 x10⁶ | 1.28 | 0 | | |
| | 5 | 5.0 x10³ | >1.0 x10⁶ | 1.34 | 0 | | |

TABLE 2

Effect of 3 % CuZn material on two types of bacteria: Staphylococcus aureus and Klebsiella pneumoniae.
24-hour contact time

| Sample: | Replicate | Recovery per test piece (CFU) | | Log₁₀ reductions | | Mean Log₁₀ reductions | |
|---|---|---|---|---|---|---|---|
| | | S. aureus ATCC 6538 | K. pneumoniae NCO9633 | S. aureus ATCC 6538 | K. pneumoniae NCO9633 | S. aureus ATCC 6538 | K. pneumoniae NCO9633 |
| Inoculum (zero time) | - | 2.8 x10⁵ | 2.4 ×10⁵ | - | - | - | - |
| 3% CuZn | 1 | <10 | <10 | >4.45 | >4.38 | >4.45 | >4.38 |
| | 2 | <10 | <10 | >4.45 | >4.38 | | |
| | 3 | <10 | <10 | >4.45 | >4.38 | | |
| | 4 | <10 | <10 | >4.45 | >4.38 | | |
| | 5 | <10 | <10 | >4.45 | >4.38 | | |

TABLE 3

Effect of 15 % CuZn material on two types of bacteria: Staphylococcus aureus and Klebsiella pneumoniae.
24-hour contact time

| Sample: | Replicate | Recovery per test piece (CFU) | | Log₁₀ reductions | | Mean Log₁₀ reductions | |
|---|---|---|---|---|---|---|---|
| | | S. aureus ATCC 6538 | K. pneumoniae NCO9633 | S. aureus ATCC 6538 | K. pneumoniae NCO9633 | S. aureus ATCC 6538 | K. pneumoniae NCO9633 |
| Inoculum (zero time) | - | 2.8 x10⁵ | 2.4 ×10⁵ | - | - | - | - |
| 15% CuZn | 1 | <10 | <10 | >4.45 | >4.38 | >4.45 | >4.38 |
| | 2 | <10 | <10 | >4.45 | >4.38 | | |
| | 3 | <10 | <10 | >4.45 | >4.38 | | |
| | 4 | <10 | <10 | >4.45 | >4.38 | | |
| | 5 | <10 | <10 | >4.45 | >4.38 | | |

For samples ‘3 % CuZn’ and ‘15 % CuZn’ both bacterial strains were seen to be reduced in number by >4 log over a 24-hour contact time. This was compared to the sample ‘0 % CuZn’, which displayed no significant antibacterial activity against either test organism.

### Example 2

Test of the antimicrobial composition on the fungus Candida albicans.

The test organism was prepared to approximately 1×10⁶ CFU/mL in 0.85 % NaCl. For each sample, five replicate test pieces were inoculated with an appropriate volume of the test organism (Table 2). The inocula were enumerated using pour-plates of Sabouraud Dextrose Agar (SDA) at the point of inoculation. The inoculated samples were then placed in an incubator at 24° C. ± 1° C. for 1, 8 or 24 hours at >95 % humidity. Following the required exposure times, the inoculated test pieces were aseptically removed to 9 mL of diluent. This was vigorously shaken to ensure thorough resuspension of any remaining test organisms. The resulting suspension was plated out in SDALT (SDA supplemented with 0.3 % soya lecithin and 3 % Tween 80). Plates were incubated at 24° C. ± 1° C. for at least five days. For the negative control, the samples were inoculated with an appropriate volume (Table 2) of sterile 0.85 % NaCl and incubated and analysed in the same way as the test samples.

TABLE 4

Sample inoculum volumes

| | |
|---|---|
| Sample | Inoculum volume |
| 0 % CuZn | 1.0 mL |
| 2 % CuZn | 300 µL |
| 3 % CuZn | 1.25 mL |

TABLE 5

Effect of 0 % CuZn material on the fungus Candida albicans.
1, 8 or 24 hour contact times with Candida albicans ATCC 10231

| Sample: | Replicate | Recovery per test piece | | | Log10 reductions | | | Log10 reductions of mean recovery | | |
|---|---|---|---|---|---|---|---|---|---|---|
| | | 1 hr | 8 hr | 24 hr | 1 hr | 8 hr | 24 hr | 1 hr | 8 hr | 24 hr |
| Inoculum (zero time) | - | 1.2 x10⁶ | | | - | | | - | | |
| Positive control | - | 9.1 x10⁵ | 9.6 x10⁵ | 1.5 x10⁶ | 0.12 | 0.10 | 0 | 0.12 | 0.10 | 0 |
| Negative control | - | <10 | <10 | <10 | - | - | - | - | - | - |
| 0 % CuZn | 1 | 5.7 x10⁵ | 4.1 x10⁵ | >10⁶ | 0.32 | 0.47 | <0.08 | 0.47 | 0.32 | <0.10 |
| | 2 | 4.0 x10⁵ | 6.0 x10⁵ | >10⁶ | 0.48 | 0.30 | <0.08 | | | |
| | 3 | 4.3 x10⁵ | 7.1 x10⁵ | 7.4 x10⁵ | 0.45 | 0.23 | 0.21 | | | |
| | 4 | 2.8 x10⁵ | 5.9 x10⁵ | >10⁶ | 0.63 | 0.31 | <0.08 | | | |
| | 5 | 3.8 x10⁵ | 5.8 x10⁵ | >10⁶ | 0.50 | 0.32 | <0.08 | | | |

TABLE 6

Effect of 2 % CuZn material on the fungus Candida albicans.
1, 8 or 24 hour contact times with Candida albicans ATCC 10231

| Sample: | Replicate | Recovery per test piece | | | Log10 reductions | | | Log10 reductions of mean recovery | | |
|---|---|---|---|---|---|---|---|---|---|---|
| | | 1 hr | 8 hr | 24 hr | 1 hr | 8 hr | 24 hr | 1 hr | 8 hr | 24 hr |
| Inoculum (zero time) | - | 3.6×10⁵ | | | - | | | - | | |
| Negative control | - | <10 | <10 | <10 | - | - | - | - | - | - |
| 2 % CuZn | 1 | 1.2 x10² | 1.0 x10¹ | <10 | 3.48 | 4.56 | >4.56 | 3.22 | >4.56 | >4.56 |
| | 2 | 1.9 x10² | <10 | <10 | 3.28 | >4.56 | >4.56 | | | |
| | 3 | 2.7 x10² | <10 | <10 | 3.13 | >4.56 | >4.56 | | | |
| | 4 | 2.4 x10² | <10 | <10 | 3.18 | >4.56 | >4.56 | | | |
| | 5 | 3.0 x10² | <10 | 1.0 x10¹ | 3.08 | >4.56 | 4.56 | | | |

TABLE 7

Effect of 3 % CuZn material on the fungus Candida albicans.
1, 8 or 24 hour contact times with Candida albicans ATCC 10231

| Sample: | Replicate | Recovery per test piece | | | Log10 reductions | | | Log10 reductions of mean recovery | | |
|---|---|---|---|---|---|---|---|---|---|---|
| | | 1 hr | 8 hr | 24 hr | 1 hr | 8 hr | 24 hr | 1 hr | 8 hr | 24 hr |
| Inoculum (zero time) | - | 1.5 x10⁶ | | | - | | | - | | |
| Negative control | - | <10 | <10 | <10 | - | - | - | - | - | - |
| 3 % CuZn | 1 | 4.3 x10⁵ | 1.0 x10¹ | <10 | 0.55 | 5.18 | >5.18 | 0.66 | >3.98 | >5.18 |
| | 2 | 3.9 x10⁵ | <10 | 1.0 x10¹ | 0.59 | >5.18 | 5.18 | | | |
| | 3 | 3.9 x10⁵ | 7.0 x10¹ | <10 | 0.59 | 4.33 | >5.18 | | | |
| | 4 | 1.7 ×10⁵ | 3.5 x10² | 1.0 x10¹ | 0.95 | 3.64 | 5.18 | | | |
| | 5 | 2.8 x10⁵ | 3.6 x10² | <10 | 0.73 | 3.62 | >5.18 | | | |

For samples ‘0 % CuZn’ no significant reduction in the numbers of Candida albicans was observed after 1, 8 or 24 hour contact times at 24° C. For sample ‘2 % CuZn’ a greater than 3 log reduction in the numbers of Candida albicans was observed after a contact time of 1 hour; a greater than 4 log reduction in the numbers of Candida albicans was observed after 8 hour or 24 hour contact times at 24° C. For sample ‘3 % CuZn’ no significant reduction in the numbers of Candida albicans was observed after a 1 hour contact time; a greater than 3 log reduction in the numbers of Candida albicans was observed after 8 hours at 24° C.; a greater than 5 log reduction in the number of Candida albicans was observed after 24 hours at 24° C.

### Example 3

Test of the antimicrobial composition on the bovine corona virus (BCV) strain L9.

For the preparation of the material, pieces of 1×1 cm were cut in sterile conditions and after a folding step transferred to an Eppendorf cup. For preparation of test virus solution, U373 cells were cultivated. For virus production, BCV strain L9 was added to the prepared monolayer. After an incubation period of 24-48 hours, cells were lysed by a rapid freeze/thaw cycle. Cellular debris was removed and the supernatant was directly used as the test virus suspension. Infectivity was determined by means of end point dilution titration using the microtitre process. The virucidal activity of the treated material was evaluated by calculating the decrease in titre in comparison with the virucidal activity of the non-treated material.

TABLE 8

Virus titres (bovine coronavirus) in the 10 fold assay with treated (novel green/white nylon copper infused fabric) and non-treated material (reference: Tork Premium Special Tucher) after 60 minutes exposure time.

| Product | Dilutions (log₁₀) | | | | | | | | | |
|---|---|---|---|---|---|---|---|---|---|---|
| | 1 | 2 | 3 | 4 | 5 | 6 | 7 | 8 | 9 | 10 |
| Copper material | ≤1.50 ± 0.00 | ≤1.50 ± 0.00 | ≤1.50 ± 0.00 | ≤1.50 ± 0.00 | ≤1.50 ± 0.00 | ≤1.50 ± 0.00 | ≤1.50 ± 0.00 | ≤1.50 ± 0.00 | ≤2.00 ± 0.44 | ≤1.50 ± 0.00 |
| Reference material | 2.83 ± 0.37 | 3.25 ± 0.33 | 2.63± 0.433 | 2.88 ± 0.37 | 3.38 ± 0.25 | 3.13 ± 0.37 | 2.88 ± 0.37 | 3.88 ± 0.37 | 2.63 ± 0.41 | 2.63 ± 0.25 |

After a contact time of 60 minutes, only one material residual virus could be measured with the novel green/white nylon copper infused fabric. In contrast, examining the non-treated materials residual virus could be detected in all cases. The following mean values resulted: ≤1.55 ± 0.04 (novel green/white nylon copper infused fabric) and 2.98 ± 0.12 (reference). A difference of 1.43 log₁₀ steps between both materials was visible based on the 10 fold determinations after 60 minutes exposure time.

1-22. (canceled)

23. An antimicrobial composition comprising a substrate and a metal component, wherein the metal component comprises chemically bonded copper and zinc and wherein the substrate comprises a dermatological composition.

24. The antimicrobial composition according to claim 23, wherein the dermatological composition is a cream, gel, lotion, spray or ointment.

25. The antimicrobial composition according to claim 23, wherein the copper and zinc form an alloy.

26. The antimicrobial composition according to claim 23, wherein the metal component further includes any metal selected from the group consisting of tin, iron, lead, zirconium, silver, gold, palladium, platinum, iridium, aluminium, nickel, tungsten, molybdenum, tantalum, titanium, iodine and/or any alloys thereof.

27. The antimicrobial composition according to claim 23, wherein the metal component further includes one or more salts of any of tin, iron, lead, zirconium, copper, zinc, silver, gold, palladium, platinum, iridium, aluminium, nickel, tungsten, molybdenum, tantalum, titanium and/or iodine.

28. The antimicrobial composition according to claim 23, wherein the metal component comprises particles measuring 3-50 µm.

29. The antimicrobial composition according to claim 23, wherein the metal component comprises: a) at least 60% copper; or b) 75%-80% copper.

30. The antimicrobial composition according to claim 23, wherein the metal component comprises 20-25% zinc.

31. The antimicrobial composition according to claim 23, wherein the metal component is interspersed throughout the substrate.

32. The antimicrobial composition according to claim 23, wherein the substrate comprises an emollient, an emulsifying agent, a petroleum-based agent, a thickening agent or a moisturising agent.

33. The antimicrobial composition according to claim 23, wherein the substrate further comprises a humectant, an antioxidant, an essential amino acid, a preservative and/or a fragrance agent.

34. The antimicrobial composition according to claim 23, wherein 2-6% of said composition by weight consists of the metal component.

35. The antimicrobial composition according to claim 23, wherein the composition further includes chelating compounds, magnesium sulphate and/or a copper peptide.

36. A method of treating the symptoms of aging and/or improving the appearance of the skin or aging associated skin conditions comprising adminsitering a composition according to claim 23 to a subject in need of treatment.

37. A method of manufacturing an antimicrobial composition comprising a substrate and a metal component, wherein the metal component comprises chemically bonded copper and zinc, and wherein the substrate comprises a dermatological composition for external use on a subject, the method comprising the steps of:
a) combining copper and zinc to produce said metal component;
b) heating the metal component to a molten state;
c) disrupting said molten state with a high velocity gas; and
d) combining the disrupted metal component with the substrate.

38. The method according to claim 37, wherein the dermatological composition is a cream, gel, lotion, spray or ointment.

39. The method according to claim 37, wherein the metal component is reduced in size prior to step (b) using a mechanical attrition process.

40. The method according to claim 37, wherein step (c) results in a powdered form of said metal component.

41. The method according to claim 37, wherein the metal component is heated at step (b) to 2000 degrees centigrade.

42. A method of treating an infection comprising applying the antimicrobial composition of claim 23 to the skin of a subject in a medical or veterinary setting.
